(19) United States
(12) Patent Application Publication
(10) Pub. No.: US 2006/0171966 A1
(43) Pub. Date: Aug. 3, 2006

(54) Variants of vibrio cholerae 01 biotype e1 tor with attributes of classical biotype

(75) Inventors: David A Sack, Dhaka (BD); G Balarish Nair, Dhaka (BD); S.M Faruque, Dhaka (BD)

Correspondence Address: VENABLE LLP, P.O. BOX 34385, WASHINGTON, DC, 20045-9998, US

(73) Assignee: Centre for Health and Population Research, Dhaka 1000 (BD)

(21) Appl. No.: 10/526,079

(22) PCT Filed: Aug. 29, 2003
(86) PCT No.: PCT/US03/26968

§ 371 Date: Feb. 17, 2006

Related U.S. Application Data
(60) Provisional application No. 60/406,917, filed on Aug. 30, 2002.

(51) Int. Cl. A61K 39/106; C12N 1/20

(52) U.S. Cl. 424/261.1; 435/252.3

(57) ABSTRACT

The invention relates to novel types of Vibrio cholerae that are useful for vaccines and immunological compositions.

### BACKGROUND OF THE INVENTION

1. Field of the Invention

The invention relates to novel types of Vibrio cholerae that are useful for vaccines and immunological compositions.

2. Background Information

New epidemic strains of toxigenic Vibrio cholerae have appeared at least twice in recent human history (10). Strains of the classical biotype, which had probably been responsible for most of the epidemic disease in the 19^{th} century and much of the 20^{th} century, were largely replaced as the predominant cause of epidemic cholera by strains of the El Tor biotype in most of the regions where cholera is endemic, beginning in 1961. However, the classical biotype strains reemerged as a predominant epidemic strain in parts of Bangladesh in 1982 (8, 25) and coexisted with the El Tor strains, causing disease until 1993. A second new epidemic strain, carrying the O139 rather than the O1 antigen, emerged in southern Asia in 1992 (7, 24). The O139 and El Tor O1 strains continue to cause epidemics of cholera, and there are indications that the incidence of cholera due to the O139 serogroup is on the rise in parts of India and Bangladesh.

The classical and El Tor biotypes of V. cholerae are closely related in their O-antigen biosynthetic genes (21, 31), although these two biotypes differ in many other regions of their genomes (2, 16, 17, 29, 30). Thus, O1 El Tor strains might have arisen following transfer of O1 antigen biosynthetic genes into a previously unknown environmental strain. Conversely, O139 and O1 El Tor strains are closely related in most parts of their genomes, but carry different O-antigen genes, suggesting the transfer of O139-specific genes from an unknown donor into a recipient El Tor strain (3, 28). Similar conclusions about gene transfer have emerged from comparisons of serogroups and sequences of diagnostic housekeeping genes of nonepidemic isolates (2).

### SUMMARY OF THE INVENTION

The present inventors have identified a new variety of V. cholerae O1 that appears to be a hybrid of the classical and El Tor biotypes from hospitalized patients with acute diarrhea. The phenotypic strains that distinguish the classical and El Tor biotypes of V. chlolerae O1 and important discriminating genotypic characteristics of the existence of such novel strains make them ideal for the development of new cholera vaccines.

Three new types of Vibrio cholerae O1 (designated Matlab I, Matlab II, and Matlab III) have been isolated from cholera patients and characterized. These include 24 new strains, 2 of which are Matlab I, 1 of which is Matlab II, and 21 of which are Matlab III. Phenotypic traits characterized included serotype (Inaba, Ogawa), Voges Proskauer test, Polymyxin B sensitivity, chicken cell agglutination, and sensitivity to Group IV and Group V phages. Genotypic traits were analyzed using tcpA and ctxA PCR, and acfB and rstT probes. From their phenotypic traits, Matlab I, II and III appear to be hybrids of classical and El Tor biotypes.

The invention provides isolated strains and biologically pure cultures of the Matlab I, II, and III, vaccines and pharmaceutical compositions containing them, and a method of immunization against V. cholerae.

As used herein, a culture of V. cholerae is considered to be biologically pure if essentially all of the cholera organisms in the culture or products of the culture are from one strain or type. All colonies grown from the original culture should be identical to the original taking into account the possibility that a rare mutant strain might arise from the original strain. A mutant might theoretically be detected at a frequency of less than 10⁻⁸ and these would not be detected when growing the strain using normal bacteriological procedures in which subcultures are prepared from the original.

Representative strains of Matlab I, II and m were deposited at the National Collection of Type Cultures, London, UK, on Aug. 27, 2002 under accession nos. NC13269-01, NC13270-01 and NC13271-01.

Vaccines and pharmaceutical compositions of the invention can be prepared by any acceptable method. Formulation of cholera vaccines is familiar to those of skill in the art. In one embodiment, the vaccine contains heat- or formalin-killed whole cells selected from different biotypes and serotypes of Cholera in a total dose of 10¹¹ cells per dose. In a preferred embodiment, the vaccine includes previously known strains of cholera, including O139, as well as the strains of the invention. Optionally, the vaccine may include the cholera B subunit. The killed cells may be suspended in a pharmaceutically acceptable aqueous solution, including additional carriers, excipients and adjuvants, as will be known to persons of skill in the art. Techniques and formulations generally for use in pharmaceutical compositions and vaccines may be found in Remmington's Pharmaceutical Sciences, Meade Publishing Co., Easton, Pa. One example of such a vaccine is DUKORAL®. Similar formulations can be made using the cholera strains of the present invention.

The vaccine may also be formulated into liposomes, as known in the art, for additional immunogenicity. Means for formulating liposomal compositions are described, inter alia, by Dima et al., Arch. Microbiol. Immunol. 60(1) 27-54 (2001); Harokopakis et al., Infect. Immun. 66(9):4299-304 (1998); Kalambaheti et al., Vaccine 16(2-3):201-7 (1998); Chaicumpa et al., Vaccine 16(7):678-84 (1998); Chaicumpa et al. J. Allergy Immunol. 8(2):87-94 (1990); Chaicumpa et al., Asian Pac. J. Allergy Immunol. 6(2):70-6 (1988).

In one preferred embodiment, the method of immunization against cholera comprises administering killed whole cells of the cholera stains of the invention in an effective amount to an individual in need of protection against cholera. Most preferably, the effective amount is contained in a single dose. Two or more doses may be necessary in some cases to establish a desired level of protection. The cells may be administered by any acceptable route, preferably oral. Preferably the cells are administered in the form of a vaccine or pharmaceutical composition, as described above.

In another preferred embodiment, the method of immunization against cholera comprises administering attenuated live cells of the cholera strains of the invention in an effective amount to an individual in need of protection against cholera. Preferably, the effective amount is contained in a single dose.

The invention also includes a combination vaccine effective for immunization against the cholera strains of the invention, other known cholera strains and additional infectious organisms such as E. coli and rotavirus.

In one particularly preferred embodiment, the invention provides an isolated strain or biologically pure culture of V. cholerae having the identifying characteristics of a strain selected from the group consisting of Matlab I, Matlab II and Matlab III. The identifying characteristics may be phenotypic traits and/or genotypic traits. Most preferred is an isolated Vibrio cholerae strain having the characteristics of Matlab I, II or III, deposited at the National Collection of Type Cultures, London, UK, on Aug. 27, 2002 with the depository numbers of NC13269-01, NC13270-01 and NC13271-01, respectively.

In another particularly preferred embodiment, the invention provides a vaccine or pharmaceutical/immunological composition for protection against cholera comprising V. cholerae having the identifying characteristics of V. cholerae selected from the group consisting of Matlab I, Matlab II and Matlab III. The vaccine or composition preferably comprises killed whole cells. The cells may be killed by any method known in the vaccine arts, for example, by heat or formalin. Preferably the vaccine is an oral vaccine. In one preferred embodiment, the number of organisms per dose of said V. cholerae is between about 10⁴ and 10¹⁶. In another preferred embodiment, the strain of V. cholerae is combined with at least one additional strain of V. cholerae. The vaccine may also include a cholera toxoid. Also contemplated is a combination vaccine, which includes at least one component effective against an additional organism, such as rotavirus and enterotoxigenic E. coli. The vaccine/composition optionally includes a pharmaceutically acceptable excipient, adjuvant or carrier, preferably suitable for oral administration, such as a sterile saline buffered from about pH 7.1 to about pH 7.3.

In another particularly preferred embodiment, the invention includes a method of protecting humans against cholera comprising:

obtaining a V. cholerae culture comprising a V. cholerae having substantially all of the identifying characteristics of V. cholerae selected from the group consisting of Matlab I, Matlab II, and Matlab III; and

administering an effective amount of said culture to a human.

Preferably the culture is administered orally in a single dose.

Thus, the invention also includes the use of the strain of Matlab I, II, or III in a vaccine or immunological composition.

In yet another preferred embodiment, the invention includes an isolated strain of V. cholerae having the genotypic or genotypic characteristics of Matlab I, II, or III that has been attenuated, for example by excising the CTX prophage DNA that carries genes for cholera toxin. In this aspect, the invention includes such an isolated strain substantially does not secrete cholera toxin. Particularly preferred strains are those that are designated ______ deposited at the National Collection of Type Cultures, London, UK, on ______.

The invention further includes the use of all of the above-mentioned attenuated strains in a cholera vaccine or immunological/pharmaceutical composition. The vaccine or composition may be comprised of killed whole cells (killed, for example, by heat or formalin) or live cells, and is preferably an oral vaccine. The number of organisms per dose of said V. cholerae will generally be between about 10⁴ and 10¹⁶. the vaccine or immunological composition may also include additional strains of V. cholerae and/or a cholera toxoid and may also be a combination vaccine that includes vaccine components effective against at least one organism in addition to V. cholerae. Particularly preferred for the combination vaccine are rotavirus and enterotoxigenic E. coli.

These and other aspects of the invention will be clear to those of skill in the art from the above description and the examples set forth below.

### BRIEF DESCRIPTION OF THE DRAWING

FIG. 1 shows Bg/I restriction patterns of rRNA genes of V. Cholerae strains compared to those of selected typical strains of the El Tor and classical biotypes of V. cholerae O1. A Southern blot of Bg/I-digested genomic DNA was hybridized with the 7.5-kb BamHI fragment of E. coli rRNA clone pKK3535. Lanes (including strain designations and relevant characteristics): 1, toxigenic El Tor strain G-3669 (isolated in 1969 in Bangladesh; 2 through 10, strains MH-08 (Matlab type III), MG-116926 (Matlab type III), MG-117086 (Matlab type III), MG-116926 (Matlab type III), MG-116955 (Matlab type III), MG-116025 (Matlab type III), MG-116226 (Matlab type II), MJ-1485 (Matlab type I), and MJ-1236 (Matlab type 1); 11, toxigenic El Tor strain 1849 (isolated in 2001); 12, toxigenic classical biotype strain (isolated in 1963 in Bangladesh).

### DETAILED DESCRIPTION OF THE INVENTION

Materials and methods

Twenty four strains of V. cholerae isolated between 1991 and 1994 from hospitalized patients with acute diarrhea in the Matlab hospital, 45 km south of Dhaka, Bangladesh, were included in this study (34). The strains were isolated following standard methods of isolation of V. cholerae from stool samples which have been published in the WHO manual for isolation of enteric pathogens, and will be familiar to those of skill in the art. The basis of a retrospective examination of these strains was their unusual response to polymyxin B (50U), chicken cell agglutination (CCA), Voges-Proskauer (VP) reaction, and sensitivity to group IV and V phages, all of which are phenotypic traits commonly used to differentiate between the classical and El Tor biotypes. The 24 strains were reexamined for the above phenotypic characteristics by standard procedures.

The presence of the ctxA gene and the variants of the classical and El Tor tcpA genes were determined by a multiplex PCR assay (18). The expected size of the PCR amplicons was ascertained by electrophoresis in agarose gels. The identities of all PCT products were further verified with specific oligonucleotide probes. The probes for El Tor and classical biotype-specific CTX prophage repressor rstR were SacI-XbaI fragments of pHK1 and pHK2, respectively (19). The acfB gene probe was prepared from the PCR amplicon with previously reported acfB-specific primers (13). The rRNA gene probe consisted of a 7.5-kb BamHI fragment of Escherichia coli rRNA clone pKK3535 (5). Colony blots or Southern blots were prepared with nylon filters (Hybond; Amersham International plc., Aylesbury, UK) by standard methods (27). The probes were labeled by random priming (14) with a random-primer DNA labeling kit (Bethesda Research Laboratories, Gaithersburg, Md., USA) and [α-³²P]dCTP (3,000 Ci/mmol; Amersham). Colony blots and Southern blots were hybridized with the probes and autoradiographed as described by Faruque et al. (11-13).

### EXAMPLE 1

We examined the commonly used phenotypic traits used to distinguish between the El Tor and classical biotypes of V. cholerae and differentiated the 24 strains into three types (Table 1), which we classified as Matlab types I, II, and III. Matlab type I included two strains belonging to the Inaba serotype that were resistant to both the El Tor-specific group IV and the classical biotype specific group V phages, negative by the CCA and VP tests (both are classical traits), and resistant to polymyxin B (an El Tor trait). Matlab type II included one strain belonging to the Ogawa serotype that was sensitive to the group IV phage but showed negative responses in the CCA and VP tests and was sensitive to polymyxin B, all of which are classical biotype characteristics. Matlab type II included 21 Ogawa strains that showed the sensitivity to phages and polymyxin B typical of the El Tor biotype but were negative by the CCA and VP tests (both classical biotype traits).

TABLE 1
Phenotypic traits of Matlab types I, II, and III of toxigenic V. cholerae O1 isolated
from patients hospitalized with acute secretory diarrhea in Bangladesh

| | | | | | | | Phage sensitivity^{a} | |
|---|---|---|---|---|---|---|---|---|
| | | No. of strains | | | | | Group IV | Group V |
| | No. of | of serotype: | | | Sensitivity to poly- | | (El Tor biotype | (classical biotype |
| Type | strains | Inaba | Ogawa | VP test^{a} | myxin B (50U)^{b} | CAA^{a} | specific) | specific) |
| Matlab I | 2 | 2 | 0 | − | R | − | R | R |
| Matlab II | 1 | 0 | 1 | − | S | − | S | R |
| Matlab III | 21 | 0 | 21 | − | R | − | S | R |
| El Tor MAK757 | 1 | 0 | 1 | + | R | + | S | R |
| Classical 154 | 1 | 0 | 1 | − | S | − | R | S |

### EXAMPLE 2

We also examined the genotypes of the strains. Genotypically, all of the strains carried the ctxA gene, a constituent gene of the CTX prophage that encodes cholera toxin (CT), and acfB and tcpA, which are located in different gene clusters (acr and tcp gene clusters) on the V. cholerae pathogenicity island. The type I strains appeared to belong more to the classical biotype because they carried the tcpA gene and the CTX prophage repressor gene (rstR) of the classical type (Table 2). The tcpA gene of the single type II strain was of the classical type, while the rstR gene was of the El Tor type. The six representative strains of V. cholerae representing Matlab III also carried the tcpA gene of the classical type. Five of the strains had the El Tor-type rstR gene, while one carried both the El Tor and classical rstR types.

TABLE 2

| | Matlab | Yr of | tcpA | ctxA | acfB | rstR |
|---|---|---|---|---|---|---|
| Strain | type | isolation | PCR | PCR | (probe) | (probe) |
| MJ-1236 | I | 1994 | C | + | + | C |
| MJ-1485 | I | 1994 | C | + | + | C |
| MG-116226 | II | 1991 | C | + | + | E |
| MG-116025 | III | 1991 | C | + | + | E |
| MG-116955 | III | 1991 | C | + | + | E |
| MG-116926 | III | 1991 | C | + | + | E, C |
| MG-117086 | III | 1991 | C | + | + | E |
| MG-117159 | III | 1991 | C | + | + | E |
| MH-08 | III | 1992 | C | + | + | E |
| MAK757 (El Tor) | Ref | 1937 | E | + | + | E |
| 154 (classical) | Ref | UK | C | + | + | C |

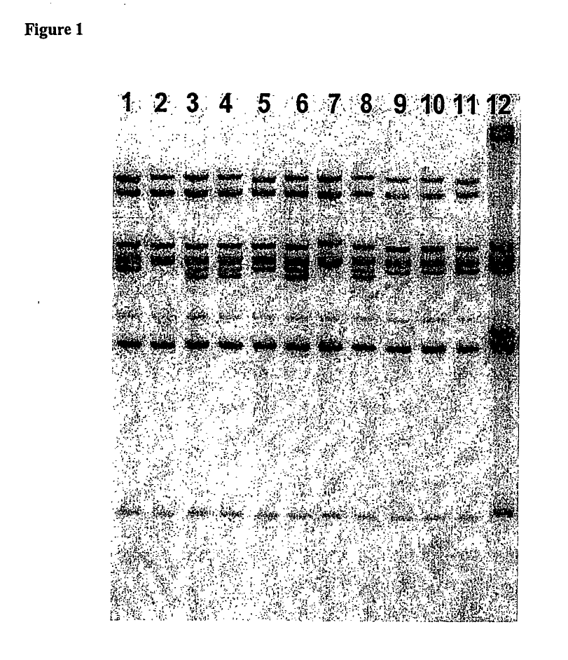

The ribotypes of the V. cholerae strains examined, compared to those of selected reference strains of the El Tor and classical biotypes, are shown in FIG. 1. The ribotypes of different strains representing the three Matlab types of V. cholerae were similar to the ribotypes of El Tor biotype strains and different from that of typical classical biotype strains described previously (11, 12). The ribotypes of two type I strains (lanes 9 and 10) were similar to that of toxigenic El Tor strains 1849 (lane 11), isolated in 2001, and G-3669 (lane 1) isolated in 1969 in Bangladesh. The Matlab type III strains belonged to three different ribotypes (FIG. 1, lanes 2 through 7), and the single type II strain had the same ribotype as a type III strain.

Classical and El Tor strains of V. cholerae are closely related but are not directly derived from each other (16, 17). El Tor vibrios appeared in Bangladesh, causing the first significant outbreak in 1968, and by 1973, they completely replaced the classical vibrios (1). In 1982, the classical biotype reappeared as the predominant epidemic strain in Bangladesh (25). In retrospect, it appears that classical cholera did not completely disappear from Bangladesh during the 1970s or late 1980s, but rather, its frequency varied in different regions of the country (26). The classical and El Tor biotypes have temporally overlapped over a decade and are likely to have interacted and exchanged genetic material either in the human intestinal milieu or in the aquatic environment. The strains isolated in this study probably represent an amalgam of such an exchange. It is well recognized that genetic exchange between divergent bacterial lineages can contribute importantly to the success of a species in complex and inconstant environments, such as those in which V. cholerae may reside. Several studies have also pointed to such exchanges as an important factor in V. cholerae population genetics and evolution (2, 3, 10).

On the basis of their phenotypic and genotypic traits, Matlab type I strains appeared to be more like the classical biotype while Matlab type II and III strains appeared to be more like the El Tor biotype. Matlab I strains, however, had altered phage receptor sites, since both of the strains were resistant to group IV and V phages. We assessed the similarity of the hybrid strains with classical and El Tor biotype strains on the basis of previously described ribotype patters of classical and El Tor strains (11, 12). Ribotyping demonstrated that the Matlab I, II, and III strains showed minor differences in fragment patterns shown by the El Tor standard strains, suggesting that the hybrids originated from an El Tor-like clone. Therefore, overall, these strains were of the El Tor biotype displaying traits of the classical biotype. It has been proposed that while El Tor and classical strains are not directly derived from each other but appear to be derived from environmental nontoxigenic strains that are El Tor-like (15). Clinical strains might become classical-like in some properties simply by loss of function, and this agrees with the findings disclosed herein. While some genetic exchange has also probably occurred, it appears that the strains have evolved classical biotype properties. With a V. cholerae genomic microarray that displayed more than 93% of the predicted genes of the whole genome sequence of El Tor strain N16961, Dziejman et al. (9) showed that only seven genes were absent solely in classical strains but present in other strains, leading them to speculate that classical biotype strains may be derived for a primordial environmental strain that was more El Tor-like than previously thought. Mitra et al. have previously reported the involvement of bacteriophage PS166 in the acquisition of some classical biotype-specific properties. By El Tor strains (22, 23). Insertion of lysogenic phage genomes in the bacterial chromosome leading to the activation or inactivation of certain genes or expression of new phage-encoded genes is a natural phenomenon in the origination of genetic diversity. However, the present invention suggests that the acquisition of classical properties such as classical-type tcpA and rstR genes by El Tor vibrios by conversion through phage PS166 seems unlikely. It seems more probable that more than one genetic exchange was involved in the conversion of these strains. Irrespective of the mechanism involved in the generation of the natural hybrid strains, the existence of strains showing a combination of classical and El Tor biotype properties has evolutionary and epidemiological importance.

Interestingly, all of the hybrid strains carried the tcpA gene of the classical type. Recently, the dominance of the classical type tcpA gene among environmental strains of V. cholerae has been reported (6). The primary structure of TcpA is highly conserved among V. cholerae serogroups and biotypes shown to be pathogenic to humans, with amino acid identities of nearly 100% between strains of a given biotype and about 80% between classical and El Tor biotype O1 strains (20). It is not clear, whether El Tor strains with classical tcpA are more efficient colonizers, but there is enough evidence showing that classical biotype strains elaborate abundant amounts of toxin-coregulated pilin when grown in vitro, in contrast to El Tor strains (20, 29). The strains analyzed in the present study may well represent precursors of other clones that could lead to a pandemic spread since they have all of the genetic features needed to make a V. cholerae strain pandemic. Moreover, these strains were isolated from clinical cases of acute diarrhea. These strains also represent unique natural recombinants that could be judiciously employed in the construction of live-vaccine strains since they have a combination of virulence attributes of both the classical and El Tor biotypes of V. cholerae O1.

The classical biotype of V. cholerae O1 is believed to be extinct and has not been isolated for the past several years, even in southern Bangladesh, the last of the niches where this biotype prevailed. The data disclosed herein shows the existence of El Tor strains that have lost some of the El Tor phenotypes and acquired classical biotype characteristics. Therefore, even though strains that represent the classical biotype in entirety have been completely displaced, a reservoir of the virulence gene of the classical biotype still exists in nature. Previous molecular analyses of classical strains isolated between 1961 and 1992 in Bangladesh support the contention that classical vibrios were never completely replaced in Bangladesh (11). Thus, a vaccine developed against cholera must take this into consideration and must be targeted against both biotypes, failing which the global use of a vaccine exclusively against the El Tor biotype might select against El Tor strains and favor strains carrying the classical attributes, such as those isolated in this study.

These hybrid strains of V. cholerae may be more common than currently recognized because phenotypic methods are inadequate to precisely distinguish between the two biotypes and are not routinely used in clinical microbiology laboratories. IS1004 fingerprinting has determined that an O37 strain of V. cholerae that was responsible for a large outbreak of cholera in Sudan in 1968 (32) is closely related to classical O1 strains (4). This indicates that horizontal exchange of genes has occurred not only between O1 biotypes but also between classical biotype and non-O1 strains, and the Sudan strain is a typical example of how a novel genotype can cause a large outbreak. For these reasons, vaccines comprised of the strains of the invention should be particularly valuable in preventing such outbreaks.

### EXAMPLE 3

### Construction of Non-Toxigenic V. cholerae Strains

The strains of V. cholerae serotype O1 described above were subjected to additional modifications to make them more suitable as vaccine strains, by removing the genes that encode cholera toxin, thus making them non-toxigenic. Cholera toxin deleted derivatives of toxigenic V. cholerae strains were constructed as follows. Briefly, the method involves excision of the CTX prophage DNA which carries genes for cholera toxin. In toxigenic V. cholerae, chromosomal CTX prophage DNA is often flanked by copies of a related satellite phage genome RS1 which uses CTXΦ-encoded proteins to form RS1Φ particles. Different CTX-RS1 arrays exist in toxigenic V. cholerae strains. We found that introduction of additional copies of RS1 element into toxigenic strains destabilized the chromosomal RS1-CTX array and led to excision of the integrated CTX prophage. The method consisted of superinfection of toxigenic strains with a genetically marked RS1 phage and passage of the strains in rabbit ileal loops followed by selection of strains which had lost the CTX phage as well as any unintegrated RS1 DNA.

Strains, Phages and Plasmids.

Toxigenic V. cholerae strains used were isolated from the stools of cholera patients admitted to the Matlab hospital of the ICDDR,B. The genetically marked page DNA pRS1-Km was a derivative of the replicative form (RF) DNA of RS1Φ, in which a kanamycin resistance (Kan^{R}) determinant was introduced as described by Faruque et al. (33). The genetically marked RS1 satellite phage RS1-KmΦ was a prepared from the culture supernatant of a control strain O395 transformed with pRS1-Km. This phage was used to infect recipient toxigenic V. cholerae strains by missing defined quantity of bacteria and phage and incubating at 30° C. Transductants were selected by plating the mixture on culture plates containing kanamycin (50 μg/ml).

Kan^{R} colonies were picked and grown for several generations, and then tested for lack of CTX genes by using specific probes as described later. Representative colonies were also passaged in the ileal loops of rabbits and derivatives which had lost both CTX phage and pRS1-Km were identified as follows.

Animal Experiments.

Selected colonies were grown in nutrient broth and passaged in ileal loops of adult New Zealand White rabbits obtained form the breeding facilities of ICDDR,B. Several short loops of approximately 6 to 8 cm in length were made in each rabbit which had previously been fasted for 48 hr. One ml of the cell suspension was inoculated into each loop by injection. After 18 hr., rabbits were sacrificed and the contents of the ileal loops were cultured on tarocholate-tellurite-gelatin agar (TTGA) plates. Vibrio colonies which became sensitive to kanamycin were identified and tested for the absence of CT genes by DNA hybridization and the presence of other relevant genes by PCR assays.

Probes and PCR Assays.

The gene probes used to detect the CTX phage genome were a 0.5 kb cloned fragment of the ctxA gene, an 840 bp region internal to the zot gene amplified by PCR, and a 2.1 kb SphI-XbaI fragment of pCTX-Km containing the entire zot and ace genes and part of orfU. Probes were labeled using a random primers DNA labeling kit (Invitrogen Corporation, Carlsbad, Calif.) and [α-³²P]ATP-deoxcytidine triphosphate (3,000 Ci/mmol, Amersham Biosciences, Uppsala, Sweden). Colony blots or Southern blots were prepared using nylon filters (Hybond, Amersham) and hybridized with the labeled probes following standard methods. PCR assays used in this study for different virulence associated genes included PCR assays specific for the tcpA, tcpI and acfB genes of the TCP pathogenicity island, and the rstR and rstC genes of the RS1-element. PCR reagents and kits were obtained either from Perkin-Elmer Corp. (Norwalk, Conn.) or Invitrogen Corporation and PCR was done essentially as described previously.

ELISA for CT.

Strains were also tested for lack of CT production by the G_{M1}-ganglioside dependent enzyme linked immunosorbent assay (G_{M1}-ELISA). Using a rabbit anti-CT monoclonal antibody (Sigma Chemical Company, St. Louis, Mo., USA). For each round of CT assay, 5 ml of AKI medium (1.5% Bactopeptone, 0.4% Yeast extract, 0.5 NaCl, 0.3% NaHCO₃, pH 7.4) was inoculated with approximately 1×10³ bacterial cells and grown for 16 hr at 30° C. with shaking. The culture was centrifuged at 4000×g for 5 min, and the supernatant was collected. Aliquots of the undiluted supernatant, 10 fold and 100 fold dilutions of the supernatant, and dilutions of purified CT (Sigma) were used for the toxin assay following standard methods. Two strains were selected for further genetic manipulations and the attenuated strains were labeled as Matlab I and Matlab II. All required tests were done on these genetically manipulated strains to ensure that they do not produce cholera toxin and nor do they have the genes necessary for production of cholera toxin. These attenuated strains were also tested in animal models.

References and publications cited herein are listed below for convenience and are hereby incorporated by reference.

### REFERENCES

1. Bart, K. J., Z. Huq, M. Khan, and W. H. Mosley. 1970. Seroepidemiologic studies during a simultaneous epidemic of infection with El Tor Ogawa and classical Inaba Vibrio cholerae. J. Infect. Dis. 121 (Suppl.): S17-S24.
2. Beltran, P., G. Delgado, A. Davarro, F. Trujillo, R. K. Selander, and A. Cravioto. 1999. Genetic diversity and population structure of Vibrio cholerae. J. Clin. Microbiol. 37:581-590.
3. Bik, E. M., A. E Bunschoten, R. D. Gouw, and F. R. Mooi. 1995. Genesis of the novel epidemic Vibrio cholerae O139 strain: evidence for horizontal transfer of genes involved in polysaccharide synthesis. EMBO J. 14:209-216.
4. Bik, E. M, R. D. Gouw, and F. R. Mooi. 1996. DNA fingerprinting of Vibrio cholerae strains with a novel insertion sequence element: a tool to identify epidemic strains. J. Clin. Microbiol. 34:1453-1461.
5. Brosius, J., A. Ullhrich, M. A. Raker, A. Gray, T. J. Dull, R. R. Gutell, and H. F. Noller. 1981. Construction and fine mapping of recombinant plasmid containing the rrnB ribosomal RNA operon of E. coli. Plasmid 6:112-118.
6. Chakraborty, S., A. K. Mukhopadhyay, R. K. Bhadra, et al. 2000. Virulence genes in environmental strains of Vibrio cholerae. Appl. Environ. Microbiol. 66:4022-4028.
7. Cholera Working Group. 1993. Large epidemic of cholera-like disease in Bangladesh caused by Vibrio cholerae O139 synonym Bengal. Lancet 342: 387-390.
8. Clemens, J. D, J. R. Harris, D. A. Sack, et al. 1988. Field trial of oral cholera vaccines in Bangladesh: results of one year of follow-up. J. Infect. Dis. 158:60-69.
9. Dziejman, M., E. Balon, D. Boyd, C. M. Fraser, J. F. Heidelberg, and J. J. Mekalanos. 2002. Comparative genomic analysis of Vibrio cholerae: genes that correlate with cholera endemic and pandemic disease. Proc. Nati. Acad. Sci. USA 99:1556-1561.
10. Faruque, S. M., M. J. Albert, J. J. Mekalanos. 1998. Epidemiology, genetics and ecology of toxigenic Vibrio cholerae. Microbiol. Mol. Biol. Rev. 62:1301-1314.
11. Faruque, S. M., A. R. M. A. Alim, M. M. Rahman, A. K. Siddique, R. B. Sack, and M. J. Albert. 1993. Clonal relationships among classical Vibrio cholerae O1 strains isolated between 1961 and 1992 in Bangladesh. J. Clin. Microbiol. 31,:2513-2516.
12. Faruque, S. M., M. N. Saha, Asudulghani, D. A. Sack, R. It. Sack, Y. Takeda, and G. B. Nair. 2000. The 0139 serogroup of Vibrio cholerae comprises diverse clones of epidemic and nonepidemic strains derived from multiple V. cholerae O1 and non-O1 progenitors. J. Infect. Dis. 182:1161-1168.
13. Faruque, S. M., A. K. Siddique, M. N. Saha, et al. 1999. Molecular characterization of a new ribotype of Vibrio cholerae O139 Bengal associated with an outbreak of cholera in Bangladesh. J. Clin. Microbiol. 37:130-1317.
14. Feinberg, A., and B. Vogelstein. 1984. A technique for radiolabelling DNA restriction endonuclease fragments to high specific activity. Anal. Biochem. 137:266-267.
15. Karaolis, D. K., R. Lan, and P. R. Reeves. 1994. Molecular evolution of the seventh-pandemic clone of Vibrio cholerae and its relationship to other pandemic and epidemic V. cholerae isolates. J. Bacteriol. 176:6199-6206.
16. Karaolis, D. K. R., R. Lan, and P. R. Reeves. 1995. The sixth and seventh cholera pandemics are due to independent clones separately derived from environmental, nontoxigenic, non-O1 Vibrio cholerae. J. Bacteriol. 177:3191-3198.
17. Kamolis, D. K. R., R. Lan, J. B. Kaper, and P. R. Reeves. 2001. Comparison of Vibrio cholerae pathogenicity islands in sixth and seventh pandemic strains. Infect. Immun. 69:1947-1952.
18. Kensler, S. P., and R. 1-1. Hall. 1993. Detection and biotyping Vibrio cholerae O1 with multiplex polymerase chain reaction. Lancet 341:1661.
19. Kimsey, H. H., and M. K. Waldor. 1998. CTXφ immunity: application in the development of cholera vaccines. Proc. Natl. Acad. Sci. USA 95:7035-7039.
20. Kim, T. J., M. J. Lafferty, C. M. P. Sandoe, and R. K. Taylor. 2000. Delineation of pilin domains required for bacterial association into microcolonies and intestinal colonization by Vibrio cholerae. Mol. Microbiol. 35:896-910.
21. Manning, P. A., U. H. Stroeher, and I L Morona. 1994. Molecular basis for O-antigen biosynthesis in Vibrio cholerae: Ogawa-Inaba switching, p. 77-94. In I. K. Wachsmuth, P. A. Blake, and O. Olsvik (ed.), Vibrio cholerae and cholera: molecular to global perspectives. ASM Press, Washington D.C.
22. Mitra, S. N. 1989. Mutation induced by vibriophage Ps166 infection changes biotype and phage type of Vibrio cholerae. J. Med. Microbiol. 30:137-141.
23. Mitra, S. N., R. Mukhopadhay, A- N. Ghosh, and R., K. Ghosh. 2000. Conversion of Vibrio El Tor MAK757 to classical biotype: role of phagc PS166. Virology 273:36-43.
24. Nair, G. B., T. Ramamurthy, S. K. Bhuttacharya, et al. 1994. Spread of Vibrio cholerae 0139 Bengal in India. J. Infect. Dis. 169:1029-1034.
25. Samadi, A. R., N. Shahid, A. Eusuf, M. Yunus, M. 1. Huq, M. U. Khan, A. S. M. M. Rahman, and A. S. G. Faruque. 1983. Classical Vibrio cholerae biotype displaces El Tor in Bangladesh. Lancet i:805-807.
26. Siddique, A. K., A. H. Baqui, A. Eusof, K. Haider, M. A. Hossain, I. Bashir, and K. Zaman. 1975. Survival of classic cholera in Bangladesh. Lancet 337:1125-1127.
27. Southern, E. M. 1975. Detection of specific sequences among DNA fragments separated by gel electrophoresis. J. Mol. Biol. 98:503-517.
28. Stroeher, U. H., K E. Jedani, B. K. Dredge, el al. 1995. Genetic rearrangements in the rfb regions of Vibrio cholerae O1 and O139. Proc. Nacl. Acad. Sci. USA 92:10374-10378.
29. Voss, E., and S. R. Attridge. 1993. In vitro production of toxin-coregulated pili by Vibrio cholerae El Tor. Microb. Pathog. 15:255-268.
30. Wachsmuth, K., 0. Olsvik G. M. Evins, and T. Popovic. 1994. Molecular epidemiology of cholera, p. 357-370. In 1. K. Wachsmuth, P. A. Blake, and O. Olsvik (ed.), Vibrio cholerae and cholera: molecular to global perspectives. ASM Press, Washington D.C.
31. Yamasaki, S., S. Garg, G. B. Nair, and Y. Takeda. 1999. Distribution of Vibrio cholerae O1 antigen biosynthesis genes among O139 and other non-O1 serogroups of Vibrio cholerae. FEMS Microbiol. Lett. 179:115-12 1.
32. Zinnaka, Y., and C. C. Carpenter, Jr. 1972. An enterotoxin produced by noncholera vibrios. Johns Hopkins Med. J. 131:403-411.
33. Faruque, S M, Asadulghani, Kamruzzaman, M., Nandi, R K, Ghosh, A N, Nair G B, Mekalanos, J J, Sack, D A 2002. RS1 element of Vibrio cholerae can propagate horizontally as a filamentous phage exploiting the morphogenesis genes of CTXΦ. Infect. Immun. 70:163-170.
34. Nair G B, Faruque S M, Bhuiyan N A, Kamruzzaman M, Siddique A K, Sack D A. New Variants of Vibrio cholerae O1 Biotype El Tor with Attributes of the Classical Biotype from Hospitalized Patients with Acute Diarrhea in Bangladesh. J.Clin.Microbiol. 2002;40:3296-99.

1. An isolated strain of V. cholerae having the identifying characteristics of a strain selected from the group consisting of Matlab I, Matlab II and Matlab III.

2. The strain of claim 1 wherein the identifying characteristics are phenotypic traits.

3. The strain of claim 1 wherein the identifying characteristics are genotypic traits.

4. The strain of claim 1 wherein the identifying characteristics are those of Matlab I.

5. The strain of claim 1 wherein the identifying characteristics are those of Matlab II

6. The strain of claim 1 wherein the identifying characteristics are those of Matlab III.

7. An isolated Vibrio cholerae strain having the characteristics of Matlab I, II or III, deposited at the National Collection of Type Cultures, London, UK, on Aug. 27, 2002 designated as NC13269-01, NC13270-01 or NC13271-01.

8. A biologically pure culture comprising V. cholerae having the identifying characteristics of a strain selected from the group consisting of Matlab I, Matlab II and Matlab III.

9. A vaccine for protection against cholera comprising V. cholerae having the identifying characteristics of V. cholerae selected from the group consisting of Matlab I, Matlab II and Matlab III.

10. The vaccine of claim 9 that is a killed whole cell vaccine.

11. The vaccine of claim 10 wherein the cells are killed by heat.

12. The vaccine of claim 10 wherein the cells are killed by formalin.

13. The vaccine of claim 9 that is an oral vaccine.

14. The vaccine of claim 9. wherein said V. cholerae is selected from the group consisting of V. cholerae as set forth in claim 7.

15. The vaccine of claim 9, wherein the number of organisms per dose of said V. cholerae is between about 10⁴ and 10¹⁶.

16. The vaccine according to claim 9, wherein said V. cholerae is combined with at least one additional strain of V. cholerae.

17. The vaccine according to claim 9, wherein said V. cholerae is combined with a cholera toxoid.

18. The vaccine of claim 9, which is a combination vaccine.

19. The vaccine of claim 18, which includes vaccine components effective against at least one organism selected from the group consisting of rotovirus and enterotoxigenic E. coli.

20. The vaccine of claim 9, which is effective in humans.

21-47. (canceled)
